(19) United States
(12) Patent Application Publication
(10) Pub. No.: US 2023/0226020 A1
(43) Pub. Date: Jul. 20, 2023

(54) MDMA ENANTIOMERS

(71) Applicant: Mind Medicine, Inc., New York, NY (US)

(72) Inventors: Robert BARROW, Madison, WI (US); Daniel R. KARLIN, New York, NY (US)

(73) Assignee: Mind Medicine, Inc., New York, NY (US)

(21) Appl. No.: 18/186,764

(22) Filed: Mar. 20, 2023

Related U.S. Application Data
(63) Continuation-in-part of application No. 17/734,100, filed on May 1, 2022.
(60) Provisional application No. 63/184,703, filed on May 5, 2021.

(51) Int. Cl. A61K 31/36; A61K 47/55; A61P 25/22; A61P 25/28

(52) U.S. Cl. CPC A61K 31/36; A61K 47/55; A61P 25/22; A61P 25/28

(57) ABSTRACT

A composition for use in psychotherapeutic or medical treatment of an R(−) enantiomer of MDMA or MDA. A method of treating an individual for a medical condition (especially autism and social anxiety disorders), by administering an effective amount of a composition of an R(−) enantiomer of MDMA or MDA and treating the individual. A method of reducing neurotoxicity of MDMA and MDA, by administering an effective amount of a composition of an R(−) enantiomer of MDMA or MDA to an individual and reducing neurotoxicity of MDMA or MDA while treating the individual. A method of reducing hyperthermia of MDMA and MDA. A method of reducing physical dependence or abuse liability of MDMA and MDA.

### BACKGROUND OF THE INVENTION

### 1. Technical Field

The present invention relates to compositions and methods for providing psychiatric treatment with MDMA and MDA. More specifically, the present invention relates to compositions and methods for providing safer treatment with enantiomers of MDMA and MDA.

### 2. Background Art

3,4-Methylenedioxymethamphetamine (MDMA) is a psychoactive drug that alters mood and perception, and is investigated as an adjunct in psychotherapy for posttraumatic stress disorder (PTSD), social anxiety, autism (Danforth, 2016; Danforth et al., 2018; Danforth et al., 2016; Mithoefer et al., 2019; Mithoefer et al., 2010; Oehen et al., 2013), and may later also be studied and used for a range of other medical conditions. Such conditions where MDMA or related substances may be useful include, but is not limited to, substance-use disorder, depression, anxiety disorder (including social anxiety), anxiety with life-threatening disease, personality disorder including narcistic and antisocial disorder, autism and other developmental disorders and obsessive-compulsive disorder. MDMA or related substances can also be used to enhance individual or couple therapy.

MDMA and related substances are thought to produce positive therapeutic long-term effects in the context of MDMA/substance-assisted psychotherapy by producing acute subjective positive mood effects that also enhance the effectiveness of psychotherapy and can be beneficial on their own. Such acute beneficial MDMA-effects include, but are not limited to, feelings of well-being, feelings of connectivity to others, feelings of increased trust, feelings of love, enhanced emotional empathy, and enhanced feelings of pro-sociality and prosocial behavior (Hysek et al., 2014; Liechti et al., 2001; Schmid et al., 2014; Vollenweider et al., 1998a).

Prior art discloses the use of substances in substance-assisted psychotherapy including MDMA, psilocybin, and LSD (Carhart-Harris et al., 2017; Liechti, 2017; Luoma et al., 2020; Nichols et al., 2017; Sessa et al., 2019; Trope et al., 2019). However, other substances may be more suitable with different therapeutic benefits/tolerability profiles. Additionally, MDMA is the only empathogen-type substance currently investigated for substance-assisted psychotherapy while psilocybin and LSD are psychedelics with a different effect profile and mode of action (Holze et al., 2020). Alternatives to MDMA have been suggested (Oeri, 2020). These alternative MDMA-like substances include many compounds that may share some similarity with MDMA based on their in vitro pharmacological profiles and based on reports of their subjective effects by recreational users (Oeri, 2020). 3,4-Methylenedioxamphetamine (MDA) is the only MDMA-like substance which has been used to assist psychotherapy in the past (Baggott et al., 2019; Yensen et al., 1976).

There are several side effects and safety concerns regarding MDMA. Abuse of MDMA can produce hyperpyrexia, neurocognitive defects, and increased rates of depression. MDMA can also be neurotoxic which limits its ability to be used chronically with repeat administration. Use of MDMA often impairs declarative memory, prospective memory, and higher cognitive skills. Neurocognitive deficits are associated with reduced SERT in the hippocampus, parietal cortex, and prefrontal cortex. EEG and ERP studies have shown localized reductions in brain activity during neurocognitive performance. Deficits in sleep, mood, vision, pain, psychomotor skill, tremor, neurohormonal activity, and psychiatric status, have also been demonstrated. These effects are seen more with higher doses or longer use. (Parrott, Neuroscience & Biobehavioral Reviews, Volume 37, Issue 8, 2013, Pages 1466-1484)

MDMA has two enantiomers, S(+)-MDMA and R(−)-MDMA. It is believed that the neurotoxicity of racemic MDMA is caused by the S(+) enantiomer, not the R(−) enantiomer due to the low efficacy of the R(−) enantiomer as a releaser of dopamine. The R(−) enantiomer also does not produce hyperthermia. The R(−) enantiomer may have a lower risk of abuse. (Pitts, et al. Psychopharmacology (2018) 235:377-392). While these effects have been shown in receptor studies and limited pre-clinical evidence, there is no evidence that this is the same in clinical studies.

There remains a need for effective treatments for psychotherapy with MDMA that also reduce negative side effects.

### SUMMARY OF THE INVENTION

The present invention provides for a composition for use in psychotherapeutic or medical treatment of an R(−) enantiomer of MDMA or MDA.

The present invention provides for a method of treating an individual for a medical condition (especially autism and social anxiety disorders), by administering an effective amount of a composition of an R(−) enantiomer of MDMA or MDA and treating the individual.

The present invention also provides for a method of reducing neurotoxicity of MDMA and MDA, by administering an effective amount of a composition of an R(−) enantiomer of MDMA or MDA to an individual and reducing neurotoxicity of MDMA or MDA while treating the individual.

### DESCRIPTION OF THE DRAWINGS

Other advantages of the present invention are readily appreciated as the same becomes better understood by reference to the following detailed description when considered in connection with the accompanying drawings wherein:

FIG. 1 is a chart of project schedule;

FIG. 2A is a graph of number of grooming episodes and FIG. 2B is a graph of grooming duration;

FIG. 3A is a graph of time spent in each arena and FIG. 3B is a graph of time spent interacting in each arena;

FIG. 4 is a graph of number of vocalizations;

FIG. 5 is a diagram of a three chamber social interaction test;

FIG. 6A is a graph of arena duration, FIG. 6B is a graph of arena frequency, FIG. 6C is a graph of interaction frequency, FIG. 6D is a graph of interaction index, and FIG. 6E is a graph of interaction duration;

FIG. 7A is a diagram of an open field test and FIG. 7B is a diagram of a locomotor activity test;

FIG. 8A is a graph of hyperactivity and FIG. 8B is a graph of distance moved;

FIG. 9A is a graph of grooming duration and FIG. 9B is a graph of number of groomings; and

FIG. 10 is a graph of an ultrasonic vocalization test.

### DETAILED DESCRIPTION OF THE INVENTION

The present invention provides for compositions of enantiomers of MDMA or MDA that are useful in psychotherapeutic or medical treatment. Most preferably, the composition is an R(−) enantiomer of MDMA or MDA. The compositions are particularly useful in treating social anxiety disorders in individuals and minimize the harmful side effects of neurotoxicity and hyperthermia and the increased risk of dependence and addiction found in the racemic mixture of MDMA or MDA.

The enantiomers of MDMA or MDA can have a high purity with a pharmaceutically acceptable profile and are physically stable.

The R(−) enantiomer MDMA or MDA can be administered in a dose of 10-1000 mg. MDMA and MDA are agonist agents that primarily release monoamines (serotonin, norepinephrine and dopamine) and possibly also oxytocin typically by interacting with the membrane monoamine transporters (serotonin, norepinephrine, or dopamine transporter) (Hysek et al., 2014; Hysek et al., 2012b; Simmler et al., 2013; Verrico et al., 2007).

The composition can also include prodrugs of enantiomers of MDMA or MDA. A “prodrug” as used herein, refers to a compound that includes a moiety attached to an active drug substance that is metabolized after administration to an individual and the compound is converted into the active drug substance. Using a prodrug allows for improving how the active drug is absorbed, distributed, metabolized, and excreted. Prodrugs can be used to prevent release of the active drug in the gastrointestinal tract upon administration so that the drug can be released more favorably elsewhere in the body. The prodrugs in the present invention can be referred to as “proMDMA”, “proMDA”, “pro-R(−)MDMA” or “pro-R(−)MDA”.

The prodrug compound includes a chemical modification to the enantiomer of MDMA or MDA, such as an amino acid covalently attached to an enantiomer of MDMA or MDA. The addition of the amino acid makes the active compound inactive mainly by preventing interaction with monoamine transporter, which is the site of action but also affecting bioavailability/rate of absorption. The amino acid can be lysine or any other amino acid such as alanine, arginine, asparagine, aspartic acid, cysteine, glutamine, glutamic acid, glycine, histidine, isoleucine, leucine, methionine, phenylalanine, proline, serine, threonine, tryptophan, tyrosine, or valine and typically attached to the amine (N)-group of MDMA or MDA and hence reducing pharmacological activity at the primary site of action (cell-membrane monoamine transporters including serotonin, dopamine and norepinephrine transporter), and also altering extent and rate of absorption and mainly releasing active substance in the circulation after absorption of the inactive compound. The amino acid can be any other natural or synthetic amino acid. The invention is described with lysine as amino acid example combined with MDMA and MDA. However, the invention can use any other amino acid covalently bound to any other MDMA-like substance via the amine group of the MDMA-like substance to form a peptide bond. Any other chemical modification can also be used.

The invention described herein describes in detail two examples of substances representing the invention regarding substance matters including lysMDMA (for lysine covalently bound to MDMA) and lysMDA (for lysine covalently bound to MDA). It should be understood that this description also applies to the enantiomer forms.

Compounds in the field of the present invention can generally be prepared in analogy to known routes such as described for lisdexamfetamine (patent numbers: WO2005032474A2, WO2006121552A2, U.S. Pat. No. 7,223,735B2, US2009234002A1, US20120157706A1, WO2017098533A2) which is derived from the combination of lysine as amino acid and dexamphetamine as psychoactive substance. Briefly, bis-N-protected lysine or another amino acid is activated at the carboxyl group by introducing a leaving group such as O-succinimide. In the present example, this activated lysine derivative is then allowed to react with a primary or secondary amine such as MDA or MDMA, respectively, to form the corresponding amide in the presence of a suitable non-protic base such as triethylamine, N-methylmorpholine or diisopropylethylamine. Tetrahydrofuran (THF) or dioxane is used as a suitable solvent, but others such as dimethylformamide (DMF) or dimethyl sulfoxide (DMSO) may also be considered. After isolation and purification, the compounds such as bis-N-protected lysMDA or lysMDMA are redissolved in a suitable solvent and treated with the corresponding conditions to allow deprotection, e.g., the use of an acid to remove tert-butoxycarbonyl (BOC) groups or hydrogen in the presence of a catalyst such as palladium on activated charcoal (Pd—C) to remove hydrogen-sensitive protecting groups. The final products can either be isolated as a salt from corresponding conditions or as their free base. An optional further purification step and/or conversion to a salt such as hydrochlorides or mesylates by known procedures will lead to the final products such as lysMDA or lysMDMA or any similar combination of an MDMA-like psychoactive substance linked with an amino acid.

Using the R(−) enantiomer allows for daily use of MDMA or MDA. The compositions are particularly useful in continual slow-release formulations, such as transdermal patches, that can provide a low dose over a long period of time. The compositions can also be administered in an intranasal spray.

The composition can also be in a liquid dosage form such as, but not limited to, suspensions, solutions, emulsions, elixirs, tinctures, sprays, syrups, gels, magmas, liniments, lotions, ointments, pastes, drops, or inhalants. The composition can be in a solid dosage form such as, but not limited to, capsules, films, lozenge, patch, powder, tablets, pellets, pills, or troches.

The compound of the present invention is administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants, and vehicles. The compounds can be administered orally, subcutaneously, or parenterally including sublingual, buccal, inhalation, intravenous, intramuscular, and intranasal administration. Implants of the compounds are also useful. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses over a period of several days, weeks or months. The treatment generally has a length proportional to the length of the disease process and drug effectiveness and the patient species being treated.

When administering the compound of the present invention orally, it will generally be formulated in an immediate release capsule, immediate release tablet, modified release capsule or tablet (including enteric coatings), solution or suspension. When administering the compound of the present invention parenterally, it will generally be formulated in a sublingual or buccal orally dissolving tablet, dissolving film, intranasal powder, intranasal solution, inhaled powder, inhaled solution, transdermal patch, transdermal patch with microneedles or other permeation enhancers, or as a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of treating an individual for a medical disorder, by administering an effective amount of a composition of an R(−) enantiomer of MDMA or MDA to the individual and treating the individual. The method can further include preventing or reducing side effects of neurotoxicity, hyperthermia and dependence/addiction experienced with racemic MDMA or MDA. Any of the prodrugs listed above can also be used.

Specifically, the compositions can be used in treating medical disorders or conditions including post-traumatic stress disorder, social anxiety, autism spectrum disorder, substance use disorder, depression, anxiety disorder, anxiety with life-threatening disease, personality disorder including narcistic or antisocial personality disorder, schizophrenia, obsessive compulsive disorder, couple therapy, enhancement of any psychotherapy by inducing feelings of well-being connectivity, trust, love, empathy, openness, and pro-sociality, and enhancing therapeutic bond in any psychotherapy of patients or neurotic/healthy subjects.

The present invention provides for a method of reducing neurotoxicity of MDMA and MDA, by administering an effective amount of a composition of an R(−) enantiomer of MDMA or MDA to an individual and reducing neurotoxicity of MDMA or MDA while treating the individual. This method allows for daily administration of the R(−) enantiomer of MDMA or MDA to the individual while avoiding the unwanted side effects of neurotoxicity of racemic MDMA or MDA. Any of the prodrugs listed above can also be used.

The present invention provides for a method of reducing hyperthermia of MDMA and MDA, by administering an effective amount of a composition of an R(−) enantiomer of MDMA or MDA to an individual and reducing hyperthermia of MDMA or MDA while treating the individual. This method allows for daily administration of the R(−) enantiomer of MDMA or MDA to the individual while avoiding the unwanted side effects of hyperthermia of racemic MDMA or MDA. Any of the prodrugs listed above can also be used.

The present invention provides for a method of reducing dependence and abuse liability of MDMA and MDA, by administering an effective amount of a composition of an R(−) enantiomer of MDMA or MDA to an individual and reducing the physical dependence and abuse liability of MDMA or MDA while treating the individual. This method allows for daily administration of the R(−) enantiomer of MDMA or MDA to the individual while avoiding the unwanted side effects of dependence/abuse liability of racemic MDMA or MDA. Any of the prodrugs listed above can also be used.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

Fragile X syndrome (FXS) is known as a monogenic cause of Autism Spectrum Disorders (ASD) and one of the most common inherited forms of intellectual disability. Patients with FXS not only suffer from intellectual disability, but also manifest core and secondary phenotypic traits of ASD such as hyperactivity, repetitive behaviors and problems with executive and language problems causing sociability impairments. The Fmr1-KO mouse has a neomycin resistance cassette replacing exon 5 of the fragile X mental retardation syndrome 1 (Fmr1) gene causing an increase in the number of CGG repeats that lead to hypermethylation of the Fmr1 gene, therefore inhibiting FMR protein production. Fmr1-KO mice are bred on a C57BL/6J background.

The fragile X mouse model Fmr1-KO presents strong ASD-like behaviors such as increased activity and hyperactivity, decreased anxiety, strong repetitive behavior as well as reduced social behavior and vocalization. The Fmr1-KO mouse model thus allows in vivo compound tests for the treatment of ASD-like behaviors.

The aim of the study is to examine the effects of R-MDMA, S-MDMA and RS-MDMA (separate racemic forms and a racemic mixture of MDMA) administered intraperitoneally in FMR1 knockout (KO) mice in comparison to vehicle treated FMR1 KO and C57BL/6J controls. Effects on general activity, repetitive behavior, social interaction and ultrasound vocalization are assessed after acute treatment.

Custom Breeding of FMR1 KO Mice

40 Fmr1 breeder pairs (JAX Strain B6.129P2-Fmr1tm1Cgr/J #003025) were purchased from Jackson laboratories and sent to QPS (homozygous females mated with hemizygous males). Breeder pairs were maintained at QPS for anticipated three breeding cycles (13 weeks in total) to obtain a total number of 96 male Fmr1 KO mice for the subsequent efficacy study.

Efficacy Study

A total of 112 male Fmr1 KO mice at an age of 10 weeks as well as 16 age- and sex-matched I C57BL/6J were randomly allocated to 7 groups of 16 animals per group (see TABLE 1).

TABLE 1

| Treatment group | Genotype | Treatment | N= |
|---|---|---|---|
| A | C57Bl/6 control | vehicle | 16 |
| B | FMR1 KO | vehicle | 16 |
| C | FMR1 KO | R-MDMA, 3 mg/kg | 16 |
| D | FMR1 KO | R-MDMA, 6 mg/kg | 16 |
| E | FMR1 KO | R-MDMA, 9 mg/kg | 16 |
| F | FMR1 KO | S-MDMA, 9 mg/kg | 16 |
| G | FMR1 KO | RS-MDMA, 9 mg/kg | 16 |

Mice received a three times treatment via intraperitoneal injection of either test compounds or vehicle (group A-D, Group allocation see TABLE 1).

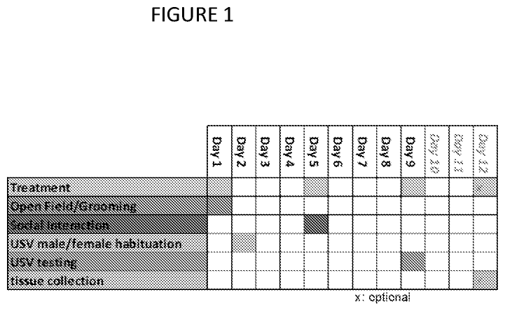

Animals were tested in the Open Field Test including grooming evaluation, Three-Chamber Social Interaction Test as well as Ultrasound vocalization after acute treatment via intraperitoneal application 30 prior to each behavioral test. Project schedule and order of behavioral tests are shown in FIG. 1.

Auto-Grooming in Fmr1-KO Mice

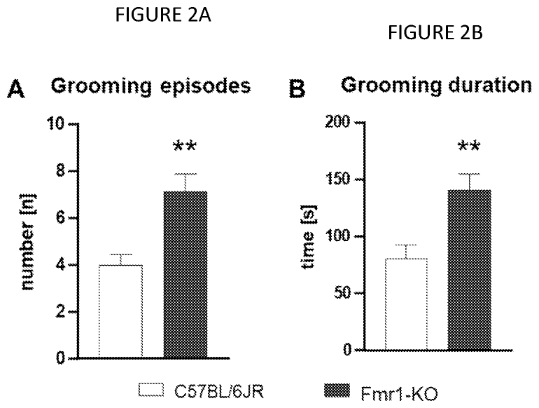

Fmr1-KO mice were tested for auto-grooming behavior at the age of 7 weeks. Parameters were measured for 10 minutes after a 5 minutes habituation phase compared to C57BL/6JRj control mice. Fmr1-KO mice presented a significantly increased number of grooming episodes (FIG. 2A) accompanied with a longer grooming duration (FIG. 2B) compared to C57BL/6JRj control mice. The effect of test articles was studied in this assay 30 minutes after administration. n=15 per group. Unpaired t-test; Mean+SEM; **p<0.01.

Social Behavior of Fmr1-KO Mice

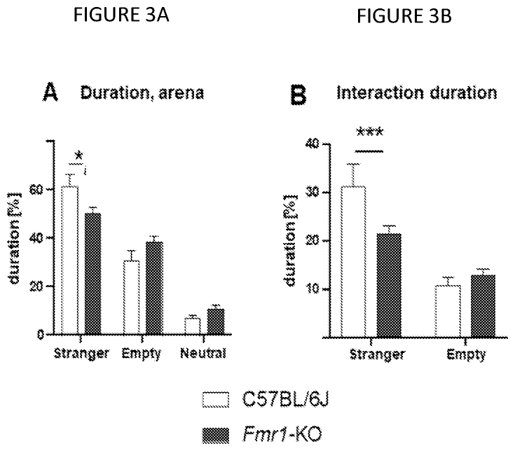

Fmr1-KO mice were tested for social behavior in the three-chamber social interaction test at the age of 7 weeks. Animals were tested in three phases: habituation, social approach, and social novelty. Social approach phase: A same sex adult stranger mouse was placed in a chamber in one of the outer arenas, while the other one stayed empty. Animals were allowed to freely explore all parts of the chamber for 10 minutes. Fmr1-KO mice spent significantly less time with the stranger mouse compared to C57BL/6JRj control mice (FIG. 3A). Additionally, the time directly interacting with the stranger mouse was reduced in Fmr1-KO mice (FIG. 3B). Results suggest a reduced social approach behavior of Fmr1-KO mice. The effect of test articles was studied in this assay 30 minutes after administration. n=15 per group. Unpaired t-test; Mean+SEM; **p<0.01; ***p<0.001.

Ultrasonic Vocalization (USV) of Fmr1-KO Mice

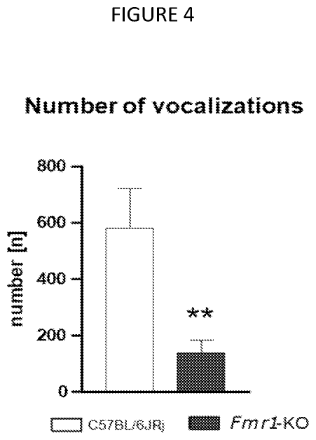

At the age of 10 weeks, ultrasonic vocalizations was recorded from male Fmr1-KO mice while they were exposed to fresh urine of estrous females. A week before testing, animals were exposed to an estrous female to familiarize them with female odor. For testing, mice were exposed to fresh urine of an estrous female while recording their USVs for 5 minutes. Fmr1-KO mice show a significantly reduced number of vocalizations (FIG. 4). The effect of test articles was studied in this assay 30 minutes after administration. n=15 per group. Unpaired t-test; Mean+SEM; **p<0.01.

### Example 2

S,R(±)-3,4-methylenedioxymethamphetamine (SR-MDMA) is a substituted phenethylamine with structural and functional similarities to amphetamine-like psychostimulants and mescaline-like hallucinogens. Several ongoing and completed clinical trials are investigating SR-MDMA as a treatment for post-traumatic stress disorder along with other conditions. Previous studies of R-MDMA in pharmacodynamics models in wild-type animals have demonstrated its potential prosocial effects and reduced locomotor hyperactivity, though it is unknown whether these effects would be present in animals with phenotypical traits of ASD. Applicants investigated a hypothesis of whether synthetic R(−)-3,4-methylenedioxymethamphetamine in development for autism spectrum disorder (ASD) is efficacious in an animal model of ASD due to Fragile X syndrome (FXS) in Fmr1 knockout (KO) mice. The Fmr1 KO mouse lacks FMRP protein due to a disruption in its Fmr1 gene and this mouse model is well established for the study of ASD/FXS with symptoms of social deficits and repetitive behaviors. In addition, phenotypic traits such as hyperactivity and altered anxiety are observed in this model.

Methods. The study was conducted in 10-week-old male Fmr1 KO mice. Animals were dosed per os with vehicle, R(−)-MDMA (8, 17 and 30 mg/kg) or SR-MDMA (8 mg/kg) with a minimum 3-day washout between doses. Animals were observed in the Open Field Test including a grooming evaluation, Three-Chamber Social Interaction Test as well as an Ultrasonic Vocalization (USV) test. Compounds were administered 30 minutes prior to each behavioral test. Basic statistical analysis was performed. If more than 2 groups were compared with each other, significance was calculated by One-way or Two-way analysis of variance (ANOVA) followed by the Bonferroni post hoc test. In case of non-normally distributed data, significance was calculated by Kruskal-Wallis test followed by Dunn's multiple comparisons test.

Open Field Test Including Grooming Behavior

Spontaneous activity and anxiety was assessed in the Open Field by evaluating the following parameters: hyperactivity [s], activity [s], distance [m], rearings [s], rearings [n] and grooming behavior. For that purpose, a Plexiglas Open Field (48×48 cm) in combination with a computerized video tracking system (Noldus Ethovision XT 14) was used.

The mice were brought to the room at least 45 minutes before the start of the testing. Each test session lasted for 5 minutes to check the mice's behavior in the new surroundings, as the first minutes of the Open Field test are the most suitable to display the exploration behavior of the animals. After the testing session, the number of fecal boluses was counted, as a measure of emotionality. The Open Field was cleaned with 70% Isopropanol after each mouse to get rid of odor traces. Testing was performed under standard room lighting conditions during the light phase of the circadian cycle.

Three-Chamber Social Interaction Test

To measure sociability, animals were tested in the Three Chamber Social Interaction Test. It consisted of a habituation phase, an approach phase and a recognition phase.

On the testing day, the test animal was placed in the center of the box and was allowed to explore it for ten minutes with two empty stranger mouse cylinders on each side of the box for habituation.

Social approach: After habituation, the mouse was removed from the box, the box was cleaned with 70% Isopropanol and dried with a hand fan. A sex-matched stranger mouse was placed into the mouse cylinder in the left chamber and the test animal was placed in the center of the box again for ten minutes.

Social recognition: After the social approach, mice were immediately tested for social recognition. The already known mouse stayed in the left chamber and a new mouse was placed under the cylinder in the right chamber. The test mouse was again placed in the middle chamber and, for 10 minutes, it was measured how much time it spent in each chamber and how much time it spent sniffing each mouse. Healthy mice will spend more time with the new mouse due to novelty. Socially disturbed mice might not recognize the already known mouse and spend therefore similar amounts of time with each mouse. It might also be that a socially disturbed mouse does not prefer social contact and will therefore spend most of the time in the middle chamber.

Both social approach and social recognition were recorded and analyzed by using Noldus Ethovision 8.5.

Ultrasound Vocalization

Adult male mice emit Ultrasonic Vocalization (USV) under selected experimental conditions, upon encountering a sexually receptive female or female urine. These vocal emissions are considered an index of emotionality, social interest, and motivation.

To induce the estrous cycle in female mice, mice were subcutaneously treated with 50 μl of 3-estradiol 48 hours prior to testing (time sensitive). Furthermore, females received a subcutaneous injection of 50 μl progesterone (16 mg/ml—P-0130, Sigma) 4 hours before the testing (time critical, +45 minutes possible (not shorter)). The estrous cycle was induced this way in female mice, both before familiarization of male study animals, and testing days. To familiarize the male animals with female odors, they were exposed to females in the estrous cycle for 5 minutes a week before testing.

On the test day, study animals were habituated in a separate soundproof room. Before recording, each mouse was separated from its home cage, placed in another cage that was covered with fresh bedding, and moved to the next soundproof room equipped with an UltraSoundGate Condenser Microphone (CM16; Avisoft Bioacoustics, Berlin, Germany), connected via an Avisoft UltraSoundGate 416 USB Audio device (Avisoft Bioacoustics) to a personal computer, where acoustic data were recorded with a sampling rate of 250,000 Hz in 16-bit format. The recording was started under red light conditions once a drop of fresh urine of an estrous female that was collected on a piece of filter paper was placed in the mouse cage. Vocal emission was recorded for 5 minutes.

For acoustical analysis, recordings were transferred to SASLab Pro (Avisoft Bioacoustics) and a fast Fourier transform was conducted (512 FFT length, 100% frame, Hamming window, and 75% time window overlap). For automatic parameter measurement setup, the maximum changes were set to 10 pixels with 5 ms minimum duration and 10 ms hold time. The total number of vocalizations and the latency to initiate the first call were extracted and evaluated for the whole 5 minutes of recording.

Statistics

Basic statistical analysis was performed. Raw data were analyzed in GraphPad PRISM™ 9 (GraphPad Software Inc., USA). Normality distribution of two groups was analyzed by Kolmogorov-Smirnov tests. Depending on Gaussian distribution, unpaired Student's T-test or the nonparametric Mann Whitney test was performed. If more than 2 groups were compared with each other, significance was calculated by One-way or Two-way analysis of variance (ANOVA) followed by the Bonferroni post hoc test. In case of non-normally distributed data, significance was calculated by Kruskal-Wallis test followed by Dunn's multiple comparisons test. Significance was defined as * p<0.5, ** p<0.01 and *** p<0.001. Graphs display group means and standard error of the mean (SEM) unless specified differently.

Results.

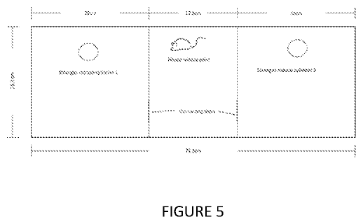

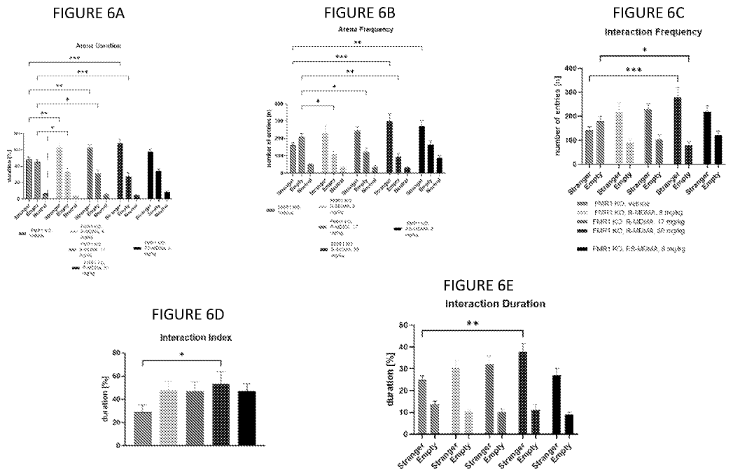

In the Three-Chamber Social Interaction Test, R(−)-MDMA dose dependently increased social interaction, measured by several parameters. For Arena Frequency, all three doses of R(−)-MDMA produced a significant difference versus vehicle treated animals in interaction with the stranger and empty chamber. Administration of SR-MDMA was not associated with significant changes. In test endpoints such as Interaction Duration, Arena Frequency and Interaction Index with the stranger were statistically significant for R(−)-MDMA at 30 mg/kg with no effect produced by SR-MDMA (8 mg/kg). During the social approach phase, treatment with test articles increased the interest of FMR1-KO mice in social interaction compared to vehicle treated FMR1-KO mice. This effect was more pronounced upon R-MDMA treatment compared to RS-MDMA treatment (FIGS. 5 and 6A-6E).

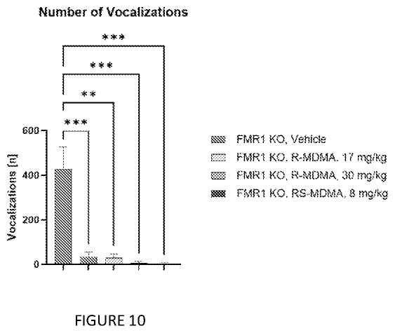

In the USV test, male mice were exposed to fresh urine from the females in estrous cycle. Vocal emission was recorded for 5 minutes and analyzed for the total number of vocalizations and the latency to initiate the first call. Treatment with R(−)-MDMA and SR-MDMA at any dose significantly reduced the number of vocalizations. Treatment with either of the test items strongly reduced social communication compared to vehicle treated FMR1-KO mice (strongly reduced number of vocalizations) (FIG. 10).

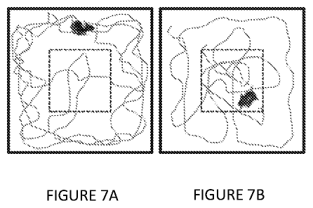

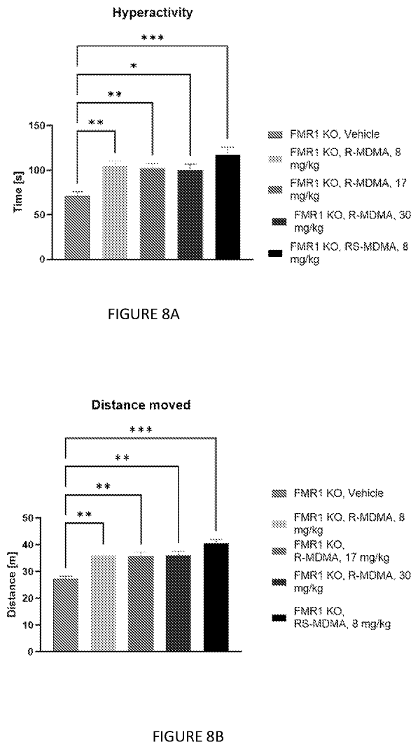

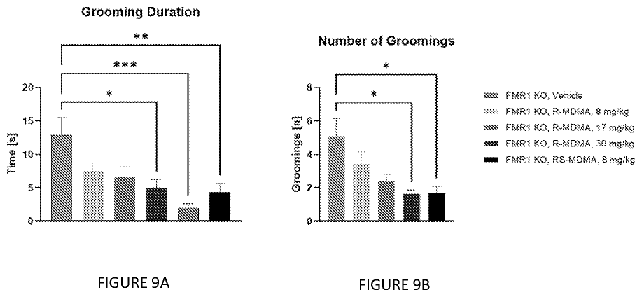

In the Open Field test, R(−)-MDMA and SR-MDMA produced hyperactivity and increases in distance moved, with R(−)-MDMA resulting in a diminished effect compared to SR-MDMA. The number of grooming occasions, a representative trait of ASD, was decreased by both compounds, with R(−)-MDMA active at 17 and 30 mg/kg and less efficacious than SR-MDMA at 8 mg/kg. Treatment of FMR1 KO mice with either of the test items increased locomotion activity compared to vehicle treated FMR1 KO mice (increased hyperactivity, distance moved), while decreasing stereotypic behavior (reduced grooming). These effects were more pronounced upon RS-MDMA treatment compared to R-MDMA (FIGS. 7A-7B, 8A-8B, and 9A-9B).

Conclusion. This study demonstrated that administration of R(−)-MDMA increased social interaction of Fmr1 KO mice, one of the characterized preclinical models of ASD/FXS. R(−)-MDMA exhibited a robust effect on social interaction and was more potent than SR-MDMA with reduced hyperactivity effects. Both compounds suppress USV in male mice equipotently.

The results of this study support clinical development of R(−)-MDMA in ASD/FXS to determine whether it has similar prosocial effects in humans and the same therapeutic efficacy.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition for use in psychotherapeutic treatment comprising an R(−) enantiomer of MDMA or MDA.

2. The composition of claim 1, wherein said R(−) enantiomer of MDMA or MDA is present in an amount of 10-1000 mg.

3. The composition of claim 1, wherein said R(−) enantiomer of MDMA or MDA includes a prodrug bound thereto.

4. The composition of claim 3, wherein said prodrug is an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

5. The composition of claim 1, wherein said composition is in a continual slow-release formulation.

6. The composition of claim 1, wherein said composition is in an intranasal spray form.

7. The composition of claim 1, wherein said composition is in a liquid dosage form chosen from the group consisting of suspensions, solutions, emulsions, elixirs, tinctures, sprays, syrups, gels, magmas, liniments, lotions, ointments, pastes, drops, and inhalants.

8. The composition of claim 1, wherein said composition is in an oral dosage form chosen from the group consisting of capsules, films, lozenge, patch, powder, tablets, pellets, pills, and troches.

9. A method of treating an individual for a medical condition, including the steps of:
administering an effective amount of a composition of an R(−) enantiomer of MDMA or MDA; and
treating the individual.

10. The method of claim 9, wherein said administering step is further defined as administering 10-1000 mg of the R(−) enantiomer of MDMA or MDA.

11. The method of claim 9, wherein said administering step is further defined as administering the R(−) enantiomer of MDMA or MDA daily.

12. The method of claim 9, further including the step of preventing or reducing side effects of neurotoxicity, hyperthermia, and dependence/addiction experienced with racemic MDMA or MDA.

13. The method of claim 9, wherein the R(−) enantiomer of MDMA or MDA includes a prodrug bound thereto.

14. The method of claim 13, wherein the prodrug is an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

15. The method of claim 9, wherein the composition is in a continual slow-release formulation.

16. The method of claim 9, wherein the composition is in an intranasal spray form.

17. The method of claim 9, wherein the composition is in a liquid dosage form chosen from the group consisting of suspensions, solutions, emulsions, elixirs, tinctures, sprays, syrups, gels, magmas, liniments, lotions, ointments, pastes, drops, and inhalants.

18. The method of claim 9, wherein the composition is in an oral dosage form chosen from the group consisting of capsules, films, lozenge, patch, powder, tablets, pellets, pills, and troches.

19. The method of claim 9, wherein said treating step is further defined as treating a condition or disorder chosen from the group consisting of post-traumatic stress disorder, social anxiety, autism spectrum disorder, substance use disorder, depression, anxiety disorder, anxiety with life-threatening disease, personality disorder, schizophrenia, obsessive compulsive disorder, couple therapy, enhancement of any psychotherapy by inducing feelings of well-being connectivity, trust, love, empathy, openness, and pro-sociality, and enhancing therapeutic bond in any psychotherapy of patients or neurotic/healthy subjects.

20. A method of reducing neurotoxicity of MDMA and MDA, including the steps of:
administering an effective amount of a composition of an R(−) enantiomer of MDMA or MDA to an individual; and
reducing neurotoxicity of MDMA or MDA while treating the individual.

21. The method of claim 20, wherein said administering step is further defined as administering 10-1000 mg of the R(−) enantiomer of MDMA or MDA.

22. The method of claim 20, wherein said administering step is further defined as administering the R(−) enantiomer of MDMA or MDA daily.

23. The method of claim 20, wherein the R(−) enantiomer of MDMA or MDA includes a prodrug bound thereto.

24. The method of claim 23, wherein the prodrug is an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

25. A method of reducing hyperthermia of MDMA and MDA, including the steps of:
administering an effective amount of a composition of an R(−) enantiomer of MDMA or MDA to an individual; and
reducing hyperthermia of MDMA or MDA while treating the individual.

26. The method of claim 25, wherein said administering step is further defined as administering 10-1000 mg of the R(−) enantiomer of MDMA or MDA.

27. The method of claim 25, wherein said administering step is further defined as administering the R(−) enantiomer of MDMA or MDA daily.

28. The method of claim 25, wherein the R(−) enantiomer of MDMA or MDA includes a prodrug bound thereto.

29. The method of claim 28, wherein the prodrug is an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

30. A method of reducing physical dependence or abuse liability of MDMA and MDA, including the steps of:
administering an effective amount of a composition of an R(−) enantiomer of MDMA or MDA to an individual; and
reducing the physical dependence or abuse liability of MDMA or MDA while treating the individual.

31. The method of claim 30, wherein said administering step is further defined as administering 10-1000 mg of the R(−) enantiomer of MDMA or MDA.

32. The method of claim 30, wherein said administering step is further defined as administering the R(−) enantiomer of MDMA or MDA daily.

33. The method of claim 30, wherein the R(−) enantiomer of MDMA or MDA includes a prodrug bound thereto.

34. The method of claim 33, wherein the prodrug is an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.
